(19) United States
(12) Patent Application Publication
(10) Pub. No.: US 2024/0331909 A1
(43) Pub. Date: Oct. 3, 2024

(54) MAGNETIZATION DEVICE

(71) Applicant: ASAHI INTECC CO., LTD., Seto-shi (JP)

(72) Inventor: Masatomo ISHIKAWA, Seto-shi (JP)

(73) Assignee: ASAHI INTECC CO., LTD., Seto-shi (JP)

(21) Appl. No.: 18/738,077

(22) Filed: Jun. 10, 2024

Related U.S. Application Data
(63) Continuation of application No. PCT/JP2022/044749, filed on Dec. 5, 2022.

(30) Foreign Application Priority Data
Dec. 17, 2021 (JP) 2021-205354

(51) Int. Cl. H01F 13/00; H01F 27/28

(52) U.S. Cl. CPC H01F 13/00; H01F 27/28

(57) ABSTRACT

A magnetization device that magnetizes a linear member, includes: a magnetization yoke that has a first end portion and a second end portion located on an opposite side of the first end portion, and is bent such that the first end portion and the second end portion are separated from and opposed to each other, and a magnetization coil that is wound around the magnetization yoke, and generates a magnetic field between the first end portion and the second end portion by application of a voltage.

### CROSS-REFERENCE TO RELATED APPLICATIONS

This application is a continuation application of International Application No. PCT/JP2022/044749, filed Dec. 5, 2022, which claims priority to Japanese Patent Application No. 2021-205354, filed Dec. 17, 2021. The contents of these applications are incorporated herein by reference in their entirety.

### TECHNICAL FIELD

The disclosed embodiments relate to a magnetization device.

### BACKGROUND ART

A magnetization device is used when an unmagnetized magnetic material is magnetized. As the magnetization device, for example, Patent Literature 1 discloses a magnetization device that forms a permanent magnet by magnetizing a magnetic material from an outer peripheral portion of a yoke housing. Patent Literature 2 discloses a magnetization device that magnetizes an assembly composed of a plurality of unmagnetized magnet materials. In addition, Patent Literature 3 discloses a magnetization device that magnetizes a plurality of magnets supported by a magnet structure.

### CITATION LIST

### Patent Literature

Patent Literature 1: JP 2007-306714 A
Patent Literature 2: JP 2018-201018 A
Patent Literature 3: JP 2019-193404 A

### SUMMARY

### Technical Problems

On the other hand, a technique of confirming a position of a medical device inserted into a living body lumen by using magnetism generated by the magnetized medical device has been developed. When the medical device is magnetized, for example, it is considered that a portion of the medical device is arranged inside a magnetization coil and magnetized. At this time, in the medical device, there is a problem in that not only the portion arranged inside the magnetization coil but also a portion arranged outside the magnetization coil is magnetized by a magnetic flux leaked to the outside of the magnetization coil. Such a problem is common to all medical devices such as a guide wire, a catheter, and an injection needle. Hereinafter, these medical devices are also referred to as “linear members.”

The disclosed embodiments have been made to solve at least a part of the above-described problem, and are directed to provide a magnetization device capable of magnetizing a predetermined magnetization target range with high accuracy in magnetization of a linear member.

### Solutions to Problems

The disclosed embodiments have been made to solve at least a part of the above-described problems, and can be realized as the following aspects.

According to one aspect of the disclosed embodiments, a magnetization device is provided. The magnetization device is a magnetization device that magnetizes a linear member, including: a magnetization yoke that has a first end portion and a second end portion located on an opposite side of the first end portion, and is bent such that the first end portion and the second end portion are separated from and opposed to each other; and a magnetization coil that is wound around the magnetization yoke, and generates a magnetic field between the first end portion and the second end portion by application of a voltage.

### BRIEF DESCRIPTION OF DRAWINGS

FIG. 1 is an explanatory view illustrating a configuration of a magnetization device of a first embodiment.

FIGS. 2 and 3 are explanatory views illustrating a configuration of a first end portion.

FIGS. 4 and 5 are explanatory views illustrating a magnetic flux generated when a voltage is applied to a magnetization coil.

FIGS. 6 to 9 are explanatory views illustrating a first end portion viewed from a +X-axis direction side.

FIG. 10 is an explanatory view illustrating a schematic configuration of a magnetization device of a second embodiment.

FIGS. 11 and 12 are explanatory views illustrating a configuration of a first end portion.

FIG. 13 is an explanatory view illustrating a schematic configuration of a magnetization device of a third embodiment.

FIG. 14 is an explanatory view illustrating an area of a portion in which a first main body part and a second main body part overlap each other.

FIGS. 15 and 16 are explanatory views illustrating the area of the portion in which the first main body part and the second main body part overlap each other.

FIG. 17 is an explanatory view illustrating a schematic configuration of a magnetization device of a fourth embodiment.

FIG. 18 is an explanatory view illustrating an area of a portion in which a first main body part and a second main body part overlap each other.

FIG. 19 is an explanatory view illustrating a schematic configuration of a magnetization device of a fifth embodiment.

FIG. 20 is an explanatory view illustrating a schematic configuration of a magnetization device of a sixth embodiment.

FIG. 21 is an explanatory view illustrating a schematic configuration of a magnetization device of a seventh embodiment.

### DETAILED DESCRIPTION

### First Embodiment

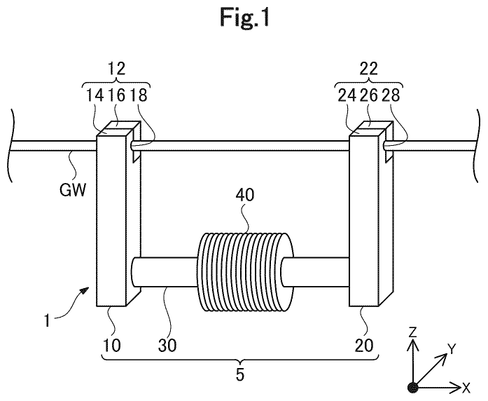

FIG. 1 is an explanatory view illustrating a configuration of a magnetization device 1 of a first embodiment. The magnetization device 1 magnetizes a guide wire GW that is a linear member. Examples of the linear member to be magnetized by the magnetization device 1 include a catheter and an injection needle in addition to the guide wire GW. The magnetization device 1 includes a magnetization yoke 5 and a magnetization coil 40. The magnetization yoke 5 has a first end portion 12 and a second end portion 22 located on the opposite side of the first end portion 12, and is bent such that the first end portion 12 and the second end portion 22 are separated from and opposed to each other, e.g., forms a U-shape. A known soft magnetic material such as permalloy, permendur, or an electromagnetic steel plate can be used for the magnetization yoke 5. In FIG. 1, X-, Y-, and Z-axes orthogonal to one another are illustrated. The X-axis corresponds to a width direction of the magnetization device 1, the Y-axis corresponds to a depth direction of the magnetization device 1, and the Z-axis corresponds to a height direction of the magnetization device 1.

The magnetization yoke 5 has a first main body part 10, a second main body part 20, and a connection portion 30. The connection portion 30 is a rod-like member that extends along the X-axis direction and connects the first main body part 10 and the second main body part 20. The magnetization coil 40 is wound in the central part of the connection portion 30 in the X-axis direction. The first main body part 10 is a bar-like member that is connected to one side of the connection portion 30 and extends along the Z-axis direction. The first main body part 10 has the first end portion 12 on the side opposite to the side connected to the connection portion 30.

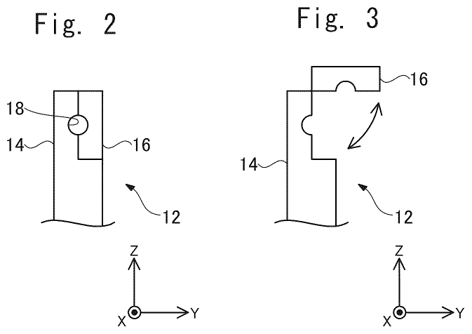

FIGS. 2 and 3 are explanatory views illustrating a configuration of the first end portion 12. In FIGS. 2 and 3, the first end portion 12 viewed from the +X-axis direction side is illustrated. In FIGS. 2 and 3, the guide wire GW is not illustrated. The first end portion 12 has a fixation portion 14, a movable portion 16, and a first accommodating part 18. As illustrated in FIGS. 2 and 3, the movable portion 16 is configured to be rotatable with respect to the fixation portion 14 by a hinge which is not illustrated.

The first accommodating part 18 is a through hole that penetrates the first end portion 12 in the X-axis direction. The first accommodating part 18 accommodates the guide wire GW that is a linear member. The term “accommodating” refers to a state in which the periphery of any transverse section of the linear member is covered. In FIG. 1, a transverse section of a portion of the guide wire GW arranged inside the first accommodating part 18 (a section in a YZ plane) is covered. As illustrated in FIGS. 2 and 3, the first accommodating part 18 is opened and closed in accordance with the rotation of the movable portion 16, and receives the guide wire GW from the outside of the magnetization device 1 in the state of FIG. 3. The guide wire GW accommodated in the first accommodating part 18 is configured to be in contact with the inner surface of the first accommodating part 18 over the entire periphery. In the present embodiment, the entire periphery of the guide wire GW is in contact with the inner surface of the first accommodating part 18. However, the present embodiment is not limited thereto, and one half or more of the entire periphery of the guide wire GW may be in contact with the inner surface of the first accommodating part 18.

Referring again to FIG. 1, the second main body part 20 is a bar-like member that is connected to the side of the connection portion 30, which is opposite to the side to which the first main body part 10 is connected, and extends along the Z-axis direction. The second main body part 20 has the second end portion 22 on the side opposite to the side connected to the connection portion 30. The second end portion 22 has a fixation portion 24, a movable portion 26, and a second accommodating part 28. The fixation portion 24 and the movable portion 26 are the same as the fixation portion 14 and the movable portion 16 in the first end portion 12. Similarly to the first accommodating part 18 in the first end portion 12, the second accommodating part 28 is a through hole that penetrates the second end portion 22 in the X-axis direction, and accommodates the guide wire GW that is a linear member. Similarly to the first accommodating part 18, the guide wire GW accommodated in the second accommodating part 28 is configured to be in contact with the inner surface of the second accommodating part 28 over the entire periphery. Regarding the second accommodating part 28, one half or more of the entire periphery of the guide wire GW may be in contact with the inner surface of the second accommodating part 28. The second accommodating part 28 accommodates a portion different from the portion that the first accommodating part 18 accommodates in an extending direction of the guide wire GW (the X-axis direction in FIG. 1). That is, in a state in which the guide wire GW is accommodated in the first accommodating part 18 and the second accommodating part 28, a portion of the guide wire GW is arranged between the first end portion 12 and the second end portion 22.

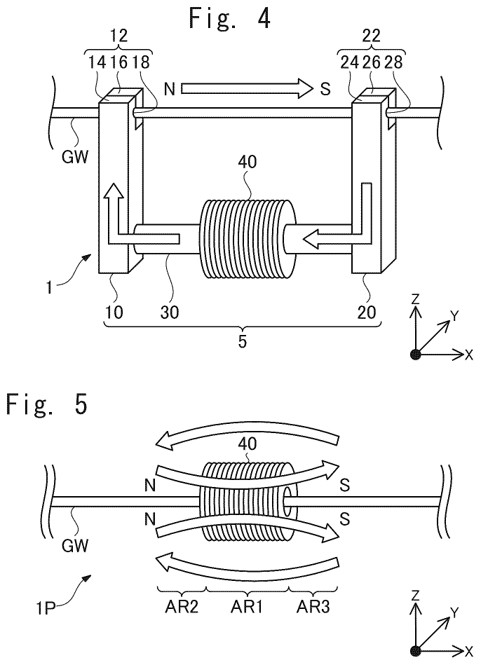

FIG. 4 is an explanatory view illustrating a direction of a magnetic flux generated when a voltage is applied to the magnetization coil 40. The magnetization coil 40 is wound around the magnetization yoke 5, and generates a magnetic field between the first end portion 12 and the second end portion 22 by application of a voltage. In FIG. 4, as indicated by white arrows, the magnetic flux passes through the portion of the guide wire GW arranged between the first end portion 12 and the second end portion 22 from one side of the magnetization coil 40 via the first main body part 10, and then is directed to the other side of the magnetization coil 40 via the second main body part 20. In this manner, when a voltage is applied to the magnetization coil 40, an annular magnetic circuit (closed magnetic circuit) is formed by the portion of the guide wire GW in a range arranged between the first end portion 12 and the second end portion 22 and the magnetization yoke 5. Since the magnetic flux flowing through such a magnetic circuit passes through the portion of the guide wire GW in the range arranged between the first end portion 12 and the second end portion 22, the portion of the guide wire GW in this range is magnetized.

FIG. 5 is an explanatory view illustrating a direction of a magnetic flux generated when a voltage is applied to a magnetization device 1P of a comparative example. The magnetization device 1P is a device that magnetizes the guide wire GW arranged in the magnetization coil 40. In FIG. 3B, as indicated by white arrows, the magnetic flux is directed from one side to the other side (+X-axis direction side) inside the magnetization coil 40, and is directed from the other side to one side (−X-axis direction side) outside the magnetization coil 40. When a voltage is applied to the magnetization coil 40, a magnetic circuit (open magnetic circuit) in which the magnetic flux that has leaked from the inside to the outside of the magnetization coil 40 returns to the inside of the magnetization coil 40 again is formed. In such a magnetization device 1P, not only a portion of the guide wire GW in a range AR1 arranged inside the magnetization coil 40 but also portions of the guide wire GW in a range AR2 and a range AR3 arranged outside the magnetization coil 40 are magnetized. Therefore, the accuracy when magnetizing a predetermined magnetization target range (for example, the range AR1) is low.

On the other hand, in the magnetization device 1 illustrated in FIG. 4, as described above, the portion of the guide wire GW in the range arranged between the first end portion 12 and the second end portion 22 is magnetized. In other words, a portion that is not arranged between the first end portion 12 and the second end portion 22 can be prevented from being magnetized. Therefore, when a distance between the first end portion 12 and the second end portion 22 is designed to be equal to the magnetization target range of the guide wire GW, a predetermined magnetization target range of the guide wire GW can be magnetized with high accuracy. In addition, as compared with the magnetization device 1P, in the magnetization device 1, the guide wire GW can be magnetized without being arranged in the magnetization coil 40, and thus the degree of freedom in device design is high.

In addition, it has been experimentally confirmed that the magnetization device 1 can magnetize the guide wire GW more strongly than the magnetization device 1P. In the magnetization device 1P illustrated in FIG. 5, it is considered that the magnetic flux directed from one side to the other side (+X-axis direction side) inside the magnetization coil 40 includes a magnetic flux passing through the guide wire GW arranged inside the magnetization coil 40 and a magnetic flux passing through a space existing between the guide wire GW and the magnetization coil 40. Therefore, it is considered that all the magnetic fluxes directed from one side to the other side (+X-axis direction side) are not concentrated on the guide wire GW. On the other hand, in the magnetization device 1 of the present embodiment illustrated in FIG. 4, it is considered that the magnetic flux directed from the first main body part 10 to the second main body part 20 is entirely concentrated on the portion of the guide wire GW arranged between the first end portion 12 and the second end portion 22. It is presumed that such concentration of the magnetic flux improves the magnetization efficiency of the guide wire GW.

As described above, according to the magnetization device 1 of the first embodiment, by applying a voltage to the magnetization coil 40, a magnetic field is generated between the first end portion 12 and the second end portion 22 which are separated from each other. Thus, when a portion of the guide wire GW is arranged between the first end portion 12 and the second end portion 22 and a voltage is applied to the magnetization coil 40, an annular magnetic circuit (closed magnetic circuit) is formed by the guide wire GW in a range arranged between the first end portion 12 and the second end portion 22 and the magnetization yoke 5. Therefore, the range of the guide wire GW arranged between the first end portion 12 and the second end portion 22 can be magnetized with high accuracy. That is, according to the magnetization device 1 of the first embodiment, a predetermined magnetization target range can be magnetized with high accuracy.

Moreover, in the present embodiment, the first end portion 12 has the first accommodating part 18 that accommodates the guide wire GW, and the second end portion 22 has the second accommodating part 28 that accommodates the guide wire GW. According to this configuration, in a state in which the guide wire GW is accommodated in the first accommodating part 18 and the second accommodating part 28, a portion of the guide wire GW in a range arranged between the first end portion 12 and the second end portion 22 can be magnetized. Therefore, the guide wire GW can be magnetized while suppressing the deviation of the distribution of the magnetic flux passing through the guide wire GW during the magnetization.

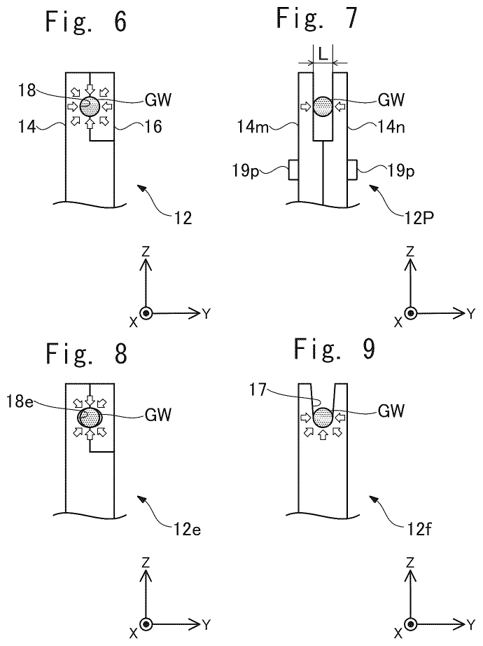

The suppression of the deviation of the distribution of the magnetic flux will be described in detail with reference to FIGS. 6 to 9. In FIG. 6, in a state in which the guide wire GW is accommodated in the first accommodating part 18, the first end portion 12 viewed from the +X-axis direction side is illustrated. On the other hand, in FIG. 7, in a state in which the guide wire GW is sandwiched by a first end portion 12P of a comparative example, the first end portion 12P viewed from the +X-axis direction side is illustrated. The first end portion 12P is configured such that a distance L between a grip portion 14m and a grip portion 14n can be adjusted by rotating an adjustment screw 19p. By adjusting the distance L, the guide wire GW is gripped between the grip portion 14m and the grip portion 14n. In FIG. 7, a portion of the periphery of the transverse section of the guide wire GW is gripped by the grip portions 14m and 14n, but the entire periphery is not covered. In such a state, when the magnetic flux flows into the guide wire GW via the first end portion 12P, the magnetic flux flows into the guide wire GW only from portions of the transverse section of the guide wire GW, which are in contact with the grip portions 14m and 14n (white arrows), thereby increasing the probability of the deviation of the distribution of the magnetic flux passing through the guide wire GW in the transverse section of the guide wire GW. On the other hand, in FIG. 6, the periphery of the transverse section of the guide wire GW is covered with the first accommodating part 18. In such a state, when the magnetic flux generated by the voltage application to the magnetization coil 40 flows into the guide wire GW via the first end portion 12, the magnetic flux flows into the guide wire GW from the entire periphery of the transverse section of the guide wire GW (white arrows), and thus the probability of the deviation of the distribution of the magnetic flux passing through the guide wire GW in the transverse section of the guide wire GW is low. Therefore, in the magnetization device 1, the guide wire GW can be magnetized while suppressing the deviation of the distribution of the magnetic flux passing through the guide wire GW. As a result, the guide wire GW uniformly magnetized over the entire periphery can be manufactured. Even in a case in which, unlike the present embodiment, the guide wire GW is not in contact with the inner surface of the first accommodating part 18 and the inner surface of the second accommodating part 28 over the entire periphery, and one half or more of the entire periphery of the guide wire GW is in contact with each of the inner surface of the first accommodating part 18 and the inner surface of the second accommodating part 28, similarly, the guide wire GW can be magnetized while suppressing the deviation of the distribution of the magnetic flux passing through the guide wire GW. For example, in a first end portion 12e illustrated in FIG. 8, although the guide wire GW is not in contact with the inner surface of a first accommodating part 18e over the entire periphery, one half or more of the entire periphery is in contact with the inner surface of the first accommodating part 18e. Moreover, in a first end portion 12f illustrated in FIG. 9, although the guide wire GW is not accommodated, one half or more of the entire periphery is in contact with the inner surface of a groove portion 17 (the first end portion 12f). As described above, even the first end portions 12e and 12f illustrated in FIGS. 8 and 9 can suppress the deviation of the distribution of the magnetic flux as compared with the first end portion 12P illustrated in FIG. 7. Needless to say, when the guide wire GW is in contact with the inner surface of the first accommodating part 18 and the inner surface of the second accommodating part 28 over the entire periphery, the deviation of the distribution of the magnetic flux can be more suppressed.

### Second Embodiment

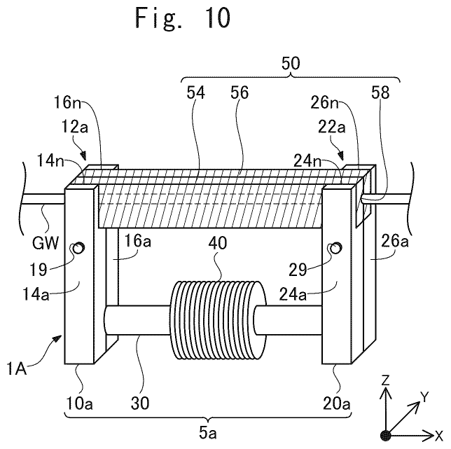

FIG. 10 is an explanatory view illustrating a schematic configuration of a magnetization device 1A of a second embodiment. The magnetization device 1A of the second embodiment is different from the magnetization device 1 of the first embodiment (FIG. 1) in that a magnetization yoke 5a different from the magnetization yoke 5 is included.

The magnetization yoke 5a includes a first main body part 10a and a second main body part 20a. The first main body part 10a includes a left bar-like member 14a, a right bar-like member 16a, and an adjustment screw 19. Similarly to the first main body part 10a, the second main body part 20a includes a left bar-like member 24a, a right bar-like member 26a, and an adjustment screw 29. In addition, the first main body part 10aand the second main body part 20a have a first end portion 12a and a second end portion 22a on the side opposite to the side connected to the connection portion 30, respectively.

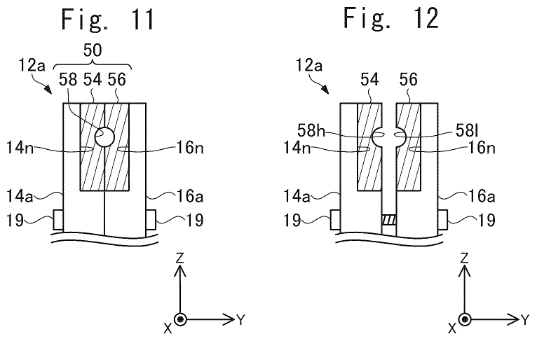

FIGS. 11 and 12 are explanatory views illustrating a configuration of the first end portion 12a. In FIGS. 11 and 12, the first end portion 12a viewed from the +X-axis direction side is illustrated. The first end portion 12a is configured such that a distance between the left bar-like member 14a and the right bar-like member 16a can be adjusted by rotating the adjustment screw 19. In FIG. 11, a state in which the distance between the left bar-like member 14a and the right bar-like member 16a is adjusted such that the left bar-like member 14a and the right bar-like member 16a are in contact with each other is illustrated. In FIG. 12, a state in which the distance between the left bar-like member 14a and the right bar-like member 16a is adjusted such that the left bar-like member 14a and the right bar-like member 16a are separated from each other is illustrated. Similarly, the second main body part 20a is configured such that a distance between the left bar-like member 24a and the right bar-like member 26a can be adjusted, e.g., translated, by rotating the adjustment screw 29.

In the first end portion 12a, a notch 14n and a notch 16n are formed in the left bar-like member 14a and the right bar-like member 16a, respectively. Similarly, in the second end portion 22a, a notch 24n and a notch 26n are formed in the left bar-like member 24a and the right bar-like member 26a, respectively (refer to FIG. 10). A left plate-like member 54 and a right plate-like member 56 are provided so as to fit into the notch 14n (24n) and the notch 16n (26n). The left plate-like member 54 and the right plate-like member 56 are plate-like members extending in the X-axis direction (refer to FIG. 10), and one end portion (−X-axis direction side in FIG. 10) is fitted into the notch 14n and the notch 16n, and the other end portion (+X-axis direction side in FIG. 10) is fitted into the notch 24n and the notch 26n. As illustrated in FIG. 12, a recessed portion 58h which extends in the X-axis direction and is recessed in the-Y-axis direction is formed on a surface of the left plate-like member 54, which faces the right plate-like member 56. On the other hand, a recessed portion 581 which extends in the X-axis direction and is recessed in the +Y-axis direction is formed on a surface of the right plate-like member 56, which faces the left plate-like member 54. As illustrated in FIG. 11, the recessed portion 58h and the recessed portion 581 form a hole 58 in a state in which the left bar-like member 14a and the right bar-like member 16a are in contact with each other. The hole 58 is a hole extending in the X-axis direction (broken line illustrated in FIG. 10), and accommodates the guide wire GW. At this time, a portion of the guide wire GW in a range accommodated in the hole 58 is restricted from moving in the YZ plane. In the magnetization device 1A, after the guide wire GW is fitted into either the recessed portion 58h or the recessed portion 581 in the state of FIG. 12, the state shifts to the state of FIG. 6A, so that the guide wire GW is accommodated in the hole 58.

The left plate-like member 54, the right plate-like member 56, and the hole 58 (the recessed portion 58h, the recessed portion 581) described above constitute a movement restricting portion 50. That is, the movement restricting portion 50 accommodates the guide wire GW by the hole 58. Therefore, since the notches 14n, 16n in the first end portion 12a and the notches 24n, 26n in the second end portion 22a accommodate the guide wire GW via the movement restricting portion 50, in the second embodiment, the notches 14n, 16n correspond to the first accommodating part that accommodates the guide wire GW, and the notches 24n, 26n correspond to the second accommodating part that accommodates the guide wire GW. Therefore, it can be said that the movement restricting portion 50 is provided from the first accommodating part to the second accommodating part, and accommodates the guide wire GW therebetween to restrict the movement of the guide wire GW. The movement restricting portion 50 is preferably made of a highly elastic material such as urethane. When the movement restricting portion 50 is made of such a material, the recessed portion 58h and the recessed portion 581 may not be formed. That is, the surface of the left plate-like member 54, which faces the right plate-like member 56, and the surface of the right plate-like member 56, which faces the left plate-like member 54, may be flat surfaces. In such a configuration, when the guide wire GW is sandwiched by the left plate-like member 54 and the right plate-like member 56, the recessed portion 58h and the recessed portion 581 (the hole 58) are formed, and the guide wire GW is accommodated in the hole 58.

Similarly to the first embodiment, according to the above-described magnetization device 1A of the second embodiment, the range of the guide wire GW arranged between the first end portion 12a and the second end portion 22a can be magnetized with high accuracy. In addition, according to the magnetization device 1A of the second embodiment, the guide wire GW is accommodated in the first accommodating part (the notches 14n, 16n) and the second accommodating part (the notches 24n, 26n) via the movement restricting portion 50 that restricts the movement of the guide wire GW. During magnetization, a linear member such as the guide wire GW tends to move by converting magnetic energy applied to the linear member into kinetic energy so as not to be magnetized. Thus, according to the magnetization device 1A of the second embodiment, since the guide wire GW is accommodated in the movement restricting portion 50, the movement of the guide wire GW due to such magnetization can be restricted. Therefore, since the conversion of magnetic energy into kinetic energy can be suppressed, the guide wire GW can be efficiently magnetized. In addition, when the range of the guide wire GW arranged between the first end portion 12a and the second end portion 22a is magnetized so as to have relatively strong magnetism, large movement and deformation of the portion of the guide wire GW in the range or damage of portions in contact with the first end portion 12a and the second end portion 22a due to the conversion of magnetic energy into kinetic energy can be suppressed. That is, the occurrence of deformation or damage in the magnetization target range can be suppressed.

### Third Embodiment

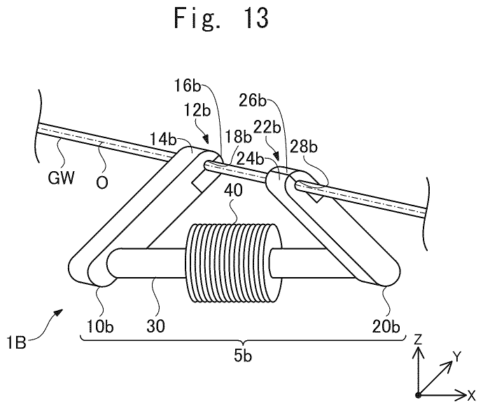

FIG. 13 is an explanatory view illustrating a schematic configuration of a magnetization device 1B of a third embodiment. The magnetization device 1B of the third embodiment is different from the magnetization device 1 of the first embodiment (FIG. 1) in that a magnetization yoke 5b different from the magnetization yoke 5 is included. In FIG. 7, an axis passing through the center of the guide wire GW is indicated by an axis line O (dash-dot line).

Each of a first main body part 10b and a second main body part 20b included in the magnetization yoke 5b is a bar-like member that extends along a direction inclined from the Z-axis direction. Similarly to the first main body part 10 of the first embodiment, the first main body part 10b has a first end portion 12b on the side opposite to the side connected to the connection portion 30. Further, as may be seen therein, ends of these member may be rounded. The first end portion 12b has a fixation portion 14b, a movable portion 16b, and a first accommodating part 18b. Similarly to the movable portion 16 of the first embodiment illustrated in FIGS. 2 and 3, the movable portion 16b is configured to be rotatable with respect to the fixation portion 14b by a hinge which is not illustrated. Similarly to the first accommodating part 18 of the first embodiment, the first accommodating part 18b is a through hole that penetrates the first end portion 12b in the X-axis direction, and accommodates the guide wire GW.

Similarly to the second main body part 20 of the first embodiment, the second main body part 20b has a second end portion 22b on the side opposite to the side connected to the connection portion 30. The second end portion 22b has a fixation portion 24b, a movable portion 26b, and a second accommodating part 28b. The fixation portion 24b and the movable portion 26b are the same as the fixation portion 14b and the movable portion 16b in the first end portion 12b. In addition, similarly to the first accommodating part 18b, the second accommodating part 28b is a through hole that penetrates the second end portion 22b in the X-axis direction, and accommodates the guide wire GW.

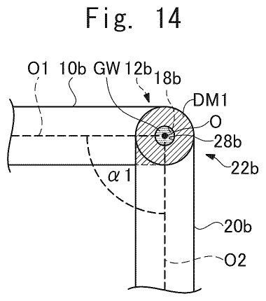

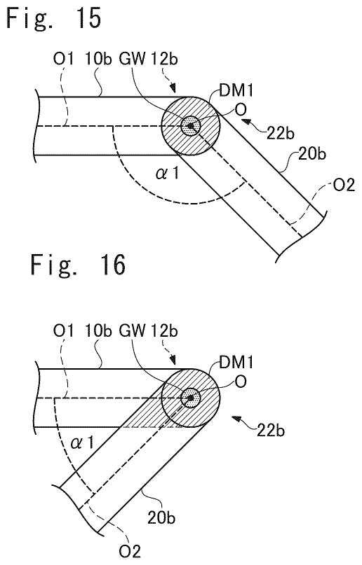

In FIG. 14, in a state in which the guide wire GW is accommodated in the first accommodating part 18b and the second accommodating part 28b (the state illustrated in FIG. 7), the first main body part 10b and the second main body part 20b viewed from an extending direction of the axis line O are illustrated. In FIG. 14, the second end portion 22b overlaps the first end portion 12b. In FIG. 8, a line indicating a boundary between the fixation portion 14b (24b) and the movable portion 16b (26b) is not illustrated for explanatory convenience (the same applies to FIGS. 15 and 16 described below). A dot-hatched portion provided inside the first accommodating part 18b and the second accommodating part 28b indicates the guide wire GW.

An angle α1 is an angle formed by a central axis O1 of the first main body part 10b and a central axis O2 of the second main body part 20b, and is 90° in FIG. 14. That is, the angle formed by the first main body part 10b and the second main body part 20bis 90°. At this time, an area DM1 (an obliquely-hatched region in FIG. 14) of a portion in which the first main body part 10b and the second main body part 20b overlap each other when the first main body part 10b and the second main body part 20b are projected in a direction in which the first end portion 12b and the second end portion 22b are separated from each other (the extending direction of the axis line O) is smaller than the area DM1 when the angle α1 is an angle equal to or more than 0° and less than 90°

FIGS. 15 and 16 illustrate the area DM1 when the angle al is an angle different from that in FIG. 8. FIG. 15 illustrates an example of the area DM1 when the angle α1 is equal to or more than 90° and less than 180°. FIG. 16 illustrates an example of the area DM1 when the angle α1 is equal to or more than 0° and less than 90°. As illustrated in FIG. 15, as the angle al decreases from 90° to 0°, an overlapping portion other than the first end portion 12b and the second end portion 22b increases, and thus the area DM1 tends to increase. On the other hand, as illustrated in FIG. 15, when the angle al is equal to or more than 90°, the first end portion 12b and the second end portion 22b mainly overlap each other, and thus the area DM1 tends to decrease. Therefore, the angle al is preferably equal to or more than 90° and equal to or less than 180°.

Similarly to the first embodiment, according to the above-described magnetization device 1B of the third embodiment, the range of the guide wire GW arranged between the first end portion 12b and the second end portion 22b can be magnetized with high accuracy. In addition, according to the magnetization device 1B of the third embodiment, the area DM1 of the portion in which the first main body part 10b and the second main body part 20b overlap each other can be made small. As the area DM1 is larger, during magnetization, the magnetic flux is more likely to leak from the first main body part 10b to the second main body part 20b without passing through the guide wire GW arranged between the first end portion 12b and the second end portion 22b. Thus, according to the magnetization device 1B of the third embodiment, since the area DM1 is small, the occurrence of leakage flux from the first main body part 10b to the second main body part 20b can be suppressed, and thus the guide wire GW can be efficiently magnetized.

### Fourth Embodiment

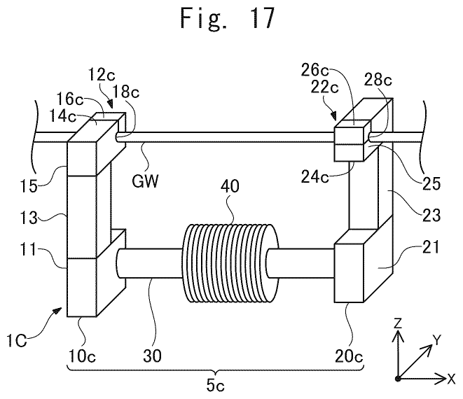

FIG. 17 is an explanatory view illustrating a schematic configuration of a magnetization device 1C of a fourth embodiment. The magnetization device 1C of the fourth embodiment is different from the magnetization device 1 of the first embodiment (FIG. 1) in that a magnetization yoke 5c different from the magnetization yoke 5 is included.

The magnetization yoke 5c includes a first main body part 10c and a second main body part 20c. The first main body part 10c has a first one end portion 11, a first intermediate portion 13, and a first other-end portion 15. The first one end portion 11 is a bar-like member that is connected to one side of the connection portion 30 and extends along the Y-axis direction. The first intermediate portion 13 is a bar-like member that is connected to the side of the first one end portion 11, which is opposite to the side to which the connection portion 30 is connected, and extends along the Z-axis direction and is narrower along the Y-axis direction that the first one end portion and the first other-end portion. The first other-end portion 15 is a bar-like member that is connected to the side of the first intermediate portion 13, which is opposite to the side to which the first one end portion 11 is connected, and extends along the Y-axis direction. The first other-end portion 15 has a first end portion 12c on the side opposite to the side connected to the first intermediate portion 13.

The first end portion 12c has a fixation portion 14c, a movable portion 16c, and a first accommodating part 18c. Similarly to the movable portion 16 of the first embodiment illustrated in FIGS. 2 and 3, the movable portion 16c is configured to be rotatable with respect to the fixation portion 14c by a hinge which is not illustrated. Similarly to the first accommodating part 18 of the first embodiment, the first accommodating part 18c is a through hole that penetrates the first end portion 12c in the X-axis direction, and accommodates the guide wire GW.

Similarly to the first main body part 10c, the second main body part 20c has a second one end portion 21, a second intermediate portion 23, and a second other-end portion 25. The second one end portion 21 is a bar-like member that is connected to the other side of the connection portion 30 and extends along the Y-axis direction. The second intermediate portion 23 is a bar-like member that is connected to the side of the second one end portion 21, which is opposite to the side to which the connection portion 30 is connected, and extends along the Z-axis direction and is narrower along the Y-axis direction that the second one end portion 21 and the second other-end portion 25. The second other-end portion 25 is a bar-like member that is connected to the side of the second intermediate portion 23, which is opposite to the side to which the second one end portion 21 is connected, and extends along the Y-axis direction. Similarly to the first other-end portion 15, the second other-end portion 25 has a second end portion 22c on the side opposite to the side connected to the second intermediate portion 23.

Similarly to the first end portion 12c, the second end portion 22c has a fixation portion 24c, a movable portion 26c, and a second accommodating part 28c. Similarly to the movable portion 16 of the first embodiment illustrated in FIGS. 2 and 3, the movable portion 26c is configured to be rotatable with respect to the fixation portion 24c by a hinge which is not illustrated. Similarly to the first accommodating part 18c, the second accommodating part 28c is a through hole that penetrates the second end portion 22c in the X-axis direction, and accommodates the guide wire GW.

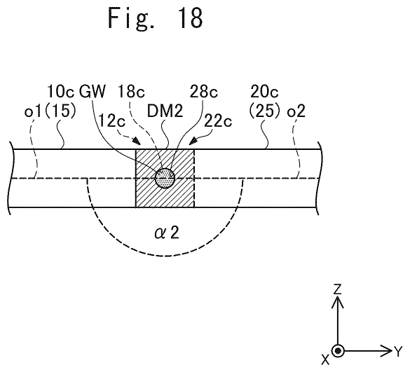

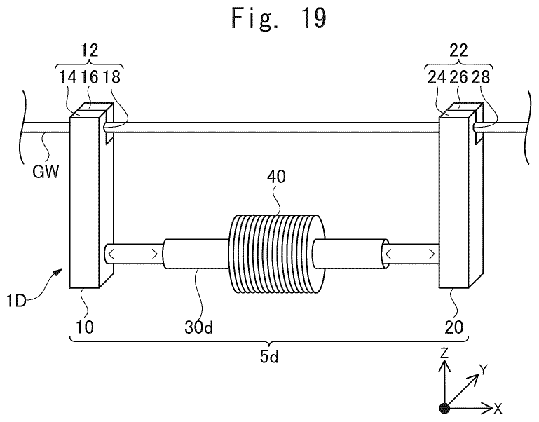

In FIG. 18, in a state in which the guide wire GW is accommodated in the first accommodating part 18c and the second accommodating part 28c (the state illustrated in FIG. 17), the first main body part 10c and the second main body part 20c viewed from the extending direction of the axis line O (+X-axis direction side in FIG. 17) are illustrated. In FIG. 18, the second end portion 22c overlaps the first end portion 12c. In FIG. 19, similarly to FIG. 14 and FIGS. 15 and 16, a line indicating a boundary between the fixation portion 14c (24c) and the movable portion 16c (26c) is not illustrated for explanatory convenience. A dot-hatched portion provided inside the first accommodating part 18c and the second accommodating part 28c indicates the guide wire GW.

An angle α2 is an angle formed by a central axis o1 of the first main body part 10c and a central axis o2 of the second main body part 20c, and is 180° in FIG. 18. That is, the angle formed by the first other-end portion 15 (the first main body part 10c) and the second other-end portion 25 (the second main body part 20c) is 180°. At this time, an area DM2 (an obliquely-hatched region in FIG. 11) of a portion in which the first main body part 10c and the second main body part 20c overlap each other when the first main body part 10c and the second main body part 20c are projected in a direction in which the first end portion 12c and the second end portion 22c are separated from each other (the extending direction of the axis line O) is smaller than the area DM2 when the angle α2 is an angle equal to or more than 0° and less than 90° (refer to FIG. 14 and FIGS. 15 and 16).

Similarly to the first embodiment, according to the above-described magnetization device 1C of the fourth embodiment, the range of the guide wire GW arranged between the first end portion 12c and the second end portion 22c can be magnetized with high accuracy. In addition, similarly to the third embodiment, according to the magnetization device IC of the fourth embodiment, the area DM2 of the portion in which the first main body part 10c and the second main body part 20c overlap each other can be made small. Therefore, the occurrence of leakage flux from the first main body part 10c to the second main body part 20c can be suppressed, and thus the guide wire GW can be efficiently magnetized.

### Fifth Embodiment

FIG. 19 is an explanatory view illustrating a schematic configuration of a magnetization device ID of a fifth embodiment. The magnetization device ID of the fifth embodiment is different from the magnetization device 1 of the first embodiment (FIG. 1) in that a magnetization yoke 5d different from the magnetization yoke 5 is included.

The magnetization yoke 5d includes a connection portion 30d different from the connection portion 30 of the first embodiment. The connection portion 30d is configured to be expandable and contractible in the X-axis direction as indicated by arrows in FIG. 19. Therefore, in the fifth embodiment, by expanding and contracting the connection portion 30d, a distance between the first main body part 10 and the second main body part 20 connected to the connection portion 30d can be adjusted. That is, a distance between the first end portion 12 and the second end portion 22 can be adjusted.

Similarly to the first embodiment, according to the above-described magnetization device ID of the fifth embodiment, the range of the guide wire GW arranged between the first end portion 12 and the second end portion 22 can be magnetized with high accuracy. In addition, according to the magnetization device ID of the fifth embodiment, since the distance between the first end portion 12 and the second end portion 22 can be freely adjusted, a magnetization target range of the guide wire GW can be adjusted to a desired range. Thus, guide wires GW having different magnetized ranges can be manufactured without preparing a plurality of magnetization devices having different separation distances between the first end portion 12 and the second end portion 22. In addition, it has been experimentally confirmed that, even when a magnetization length of the guide wire GW is increased by adjusting the magnetization target range, the magnetized range is magnetized with a constant strength regardless of the magnetization length. This is considered to be due to the fact that the amount of the magnetic flux passing through the transverse section of the guide wire GW is constant regardless of the length of the magnetization length.

### Sixth Embodiment

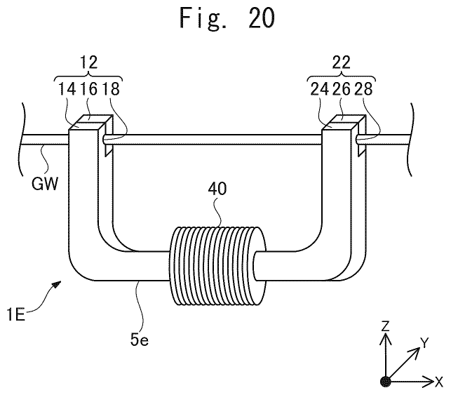

FIG. 20 is an explanatory view illustrating a schematic configuration of a magnetization device 1E of a sixth embodiment. The magnetization device 1E of the sixth embodiment is different from the magnetization device 1 of the first embodiment (FIG. 1) in that a magnetization yoke 5e different from the magnetization yoke 5 is included.

Similarly to the magnetization yoke 5 of the first embodiment, the magnetization yoke 5e has the first end portion 12 and the second end portion 22 located on the opposite side of the first end portion 12, and is bent such that the first end portion 12 and the second end portion 22 are separated from and opposed to each other. The magnetization yoke 5e is bent such that the corners thereof are rounded, e.g., forms a curved member, in which members corresponding to the first main body part 10, the second main body part 20, and the connection portion 30 of the first embodiment are integrally formed. Similarly to the first embodiment, according to the above-described magnetization device 1E of the fifth embodiment including such a magnetization yoke 5e, the range of the guide wire GW arranged between the first end portion 12 and the second end portion 22 can be magnetized with high accuracy.

### Seventh Embodiment

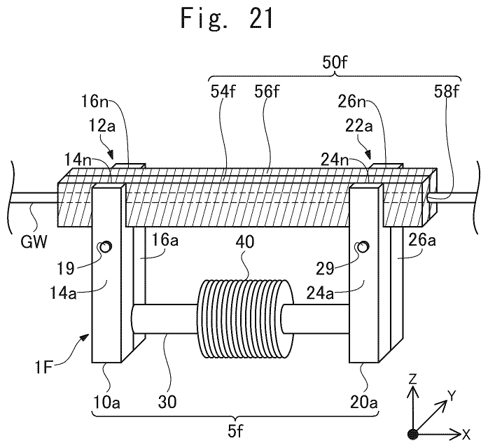

FIG. 21 is an explanatory view illustrating a schematic configuration of a magnetization device IF of a seventh embodiment. The magnetization device IF of the seventh embodiment is different from the magnetization device 1A of the second embodiment (FIG. 10) in that a magnetization yoke 5fdifferent from the magnetization yoke 5a is included.

The magnetization yoke 5fhas a movement restricting portion 50f different from the movement restricting portion 50 of the second embodiment (FIG. 10). The movement restricting portion 50f is composed of a left plate-like member 54f, a right plate-like member 56f, and a hole 58f. The left plate-like member 54f and the right plate-like member 56f are plate-like members extending in the X-axis direction, and a position closer to the +X-axis direction from one end portion (-X-axis direction side in FIG. 21) is fitted into the notch 14n and the notch 16n, and a position closer to the-X-axis direction from the other end portion (+X-axis direction side in FIG. 21) is fitted into the notch 24n and the notch 26n. Therefore, in the seventh embodiment, the left plate-like member 54f and the right plate-like member 56f extend from the outside (-X-axis direction side) of the first end portion 12a to the outside (+X-axis direction side) of the second end portion 22a along the X-axis direction. The hole 58f formed by the left plate-like member 54f and the right plate-like member 56f accommodates the guide wire GW. Similarly to the second embodiment, a portion of the guide wire GW in a range accommodated in the hole 58f is restricted from moving in the YZ plane.

Similarly to the first embodiment, according to the above-described magnetization device IF of the seventh embodiment, the range of the guide wire GW arranged between the first end portion 12a and the second end portion 22a can be magnetized with high accuracy. In addition, similarly to the second embodiment, according to the magnetization device IF of the seventh embodiment, since the guide wire GW is accommodated in the movement restricting portion 50f, the movement of the guide wire GW due to such magnetization is restricted, so that the guide wire GW can be efficiently magnetized, and the occurrence of deformation or damage in the magnetization target range can be suppressed. Furthermore, according to the magnetization device IF of the seventh embodiment, since the movement of the guide wire GW in a wider range is restricted and the conversion of magnetic energy into kinetic energy can be suppressed in a wider range as compared with the second embodiment, the guide wire GW can be efficiently magnetized equal to or more than the second embodiment.

### MODIFIED EXAMPLES OF PRESENT EMBODIMENT

The disclosed embodiments are not limited to the above embodiments, can be embodied in various aspects within a scope not departing from the spirit thereof, and can be modified as follows, for example.

### Modified Example 1

In the first to seventh embodiments described above, the configurations of the magnetization devices 1, and 1A to IF have been illustrated. However, the configuration of the magnetization device can be variously modified. For example, the magnetization device may magnetize the linear member in a state in which the linear member is arranged between the first end portion and the second end portion without accommodating the linear member. Moreover, the movement restricting portion (the second and seventh embodiments) may be a member independent of the magnetization yoke, and in this case, the movement restricting portion is fitted into the magnetization yoke during magnetization. Moreover, the movement restricting portion (the second and seventh embodiments) may be a member in which the left plate-like member, the right plate-like member, and the hole are integrally formed, and in this case, the linear member is accommodated in the movement restricting portion by being inserted from one end portion to the other end portion of the hole. Moreover, in the third embodiment (FIG. 13), the first main body part 10b and the second main body part 20b may be rotatable with respect to the connection portion 30, or may be adjustable in any direction with respect to the connection portion 30. Moreover, in the fourth embodiment (FIG. 17), members corresponding to the first one end portion 11, the first intermediate portion 13, and the first other-end portion 15 (the second one end portion 21, the second intermediate portion 23, and the second other-end portion 25) may be an integrally formed.

### Modified Example 2

The configurations of the magnetization devices 1, and 1A to IF in the first to seventh embodiments described above, and each of the configurations of the modified example 1 described above may be appropriately combined. For example, in the magnetization devices 1A and 1C to IF of the second and fourth to seventh embodiments, as described in the third embodiment, each of the first main body part and the second main body part may extend along a direction inclined from the Z-axis direction. Moreover, in the magnetization devices 1B to 1E of the third to sixth embodiments, as described in the second embodiment, the linear member may be accommodated via the movement restricting portion. Moreover, in the magnetization device ID of the fifth embodiment, as described in the fourth embodiment, each of the first and second main body parts may have the first (or second) one end portion, the first (or second) intermediate portion, and the first (or second) other-end portion. Moreover, in the magnetization devices 1A to IC and IF of the second to fourth and seventh embodiments, as described in the fifth embodiment, the connection portion may be configured to be expandable and contractible.

Although the present aspects have been described on the basis of the embodiments and the modified examples, the embodiments of the above-described aspects are made for facilitating understanding of the present aspects, and do not limit the present aspects. The present aspects can be modified and improved without departing from the spirit thereof and the claims, and include equivalents thereof. Moreover, unless the technical features are described as essential in the present description, the technical features may be appropriately omitted.

(1) According to one aspect of the disclosed embodiments, a magnetization device is provided. The magnetization device is a magnetization device that magnetizes a linear member, including: a magnetization yoke that has a first end portion and a second end portion located on an opposite side of the first end portion, and is bent such that the first end portion and the second end portion are separated from and opposed to each other; and a magnetization coil that is wound around the magnetization yoke, and generates a magnetic field between the first end portion and the second end portion by application of a voltage.

According to this configuration, by applying a voltage to the magnetization coil, a magnetic field is generated between the first end portion and the second end portion which are separated from each other. Thus, when a portion of the linear member is arranged between the first end portion and the second end portion and a voltage is applied to the magnetization coil, an annular magnetic circuit (closed magnetic circuit) is formed by the linear member in a range arranged between the first end portion and the second end portion and the magnetization yoke. Therefore, the range of the linear member arranged between the first end portion and the second end portion can be magnetized with high accuracy. That is, according to the magnetization device having this configuration, a predetermined magnetization target range can be magnetized with high accuracy.

(2) In the magnetization device of the above-described aspect, the first end portion may have a first accommodating part that accommodates the linear member, and the second end portion may have a second accommodating part that accommodates the linear member.

According to this configuration, in a state in which the linear member is accommodated in the first accommodating part and the second accommodating part, a portion of the linear member in a range arranged between the first end portion and the second end portion can be magnetized. Therefore, the linear member can be magnetized while suppressing the deviation of the distribution of a magnetic flux passing through the linear member during the magnetization.

(3) The magnetization device of the above-described aspect may further include a movement restricting portion that is provided from the first accommodating part to the second accommodating part, and accommodates the linear member from the first accommodating part to the second accommodating part to restrict movement of the linear member, in which the first accommodating part and the second accommodating part may accommodate the linear member via the movement restricting portion.

According to this configuration, the linear member is accommodated in the first accommodating part and the second accommodating part via the movement restricting portion that restricts the movement of the linear member. During magnetization, the linear member tends to move by converting magnetic energy applied to the linear member into kinetic energy so as not to be magnetized. Thus, according to this configuration, since the linear member is accommodated in the movement restricting portion, the movement of the linear member due to such magnetization can be restricted. Therefore, since the conversion of magnetic energy into kinetic energy can be suppressed, the linear member can be efficiently magnetized. In addition, when the range of the linear member arranged between the first end portion and the second end portion is magnetized so as to have relatively strong magnetism, large movement and deformation of the portion of the linear member in the range can be suppressed. That is, the occurrence of deformation in the magnetization target range can be suppressed.

(4) In the magnetization device of the above-described aspect, the magnetization yoke may include: a first main body part having the first end portion; a second main body part having the second end portion; and a connection portion that connects the first main body part and the second main body part, around which the magnetization coil is wound, and an angle formed by the first main body part and the second main body part may be equal to or more than 90° and equal to or less than 180°.

According to this configuration, the angle formed by the first main body part and the second main body part is equal to or more than 90° and equal to or less than 180°. Thus, an area of a portion in which the first main body part and the second main body part overlap each other when the first main body part and the second main body part are projected in a direction in which the first end portion and the second end portion are separated from each other can be made small. As the area of the portion in which the first main body part and the second main body part overlap each other is larger, during magnetization, the magnetic flux is more likely to leak from the first main body part to the second main body part without passing through the linear member arranged between the first end portion and the second end portion. Thus, according to this configuration, since the area is small, the occurrence of leakage flux from the first main body part to the second main body part can be suppressed, and thus the linear member can be efficiently magnetized.

(5) In the magnetization device of the above-described aspect, the connection portion may be expandable and contractible, and a distance between the first end portion and the second end portion may be adjusted by expanding and contracting the connection portion.

According to this configuration, since the distance between the first end portion and the second end portion can be freely adjusted, a magnetization target range of the linear member can be adjusted to a desired range. Thus, linear members having different magnetized ranges can be manufactured without preparing a plurality of magnetization devices having different separation distances between the first end portion and the second end portion.

The disclosed embodiments can be implemented in various aspects, such as a magnetization device, a system including a magnetization device, a device for fixing a linear member for magnetization.

What is claimed is:

1. A magnetization device that magnetizes a linear member, comprising:
a magnetization yoke that has a first end portion and a second end portion located on an opposite side of the first end portion, and is bent such that the first end portion and the second end portion are separated from and opposed to each other; and
a magnetization coil that is wound around the magnetization yoke, and generates a magnetic field between the first end portion and the second end portion by application of a voltage.

2. The magnetization device according to claim 1, wherein
the first end portion has a first accommodating part that accommodates the linear member, and
the second end portion has a second accommodating part that accommodates the linear member.

3. The magnetization device according to claim 2, further comprising:
a movement restricting portion that is provided from the first accommodating part to the second accommodating part, and accommodates the linear member from the first accommodating part to the second accommodating part to restrict movement of the linear member, wherein
the first accommodating part and the second accommodating part accommodate the linear member via the movement restricting portion.

4. The magnetization device according to claim 1, wherein
the magnetization yoke includes:
a first main body part having the first end portion;
a second main body part having the second end portion; and
a connection portion that connects the first main body part and the second main body part opposite the first end portion and the second end portion, respectively, around which the magnetization coil is wound, and
an angle formed by the first main body part and the second main body part is equal to or more than 90° and equal to or less than 180°.

5. The magnetization device according to claim 1, wherein
the connection portion is expandable and contractible, and
a distance between the first end portion and the second end portion is adjusted by expanding and contracting the connection portion.

6. The magnetization device according to claim 2, wherein
the magnetization yoke includes:
a first main body part having the first end portion;
a second main body part having the second end portion; and
a connection portion that connects the first main body part and the second main body part opposite the first end portion and the second end portion, respectively, around which the magnetization coil is wound, and
an angle formed by the first main body part and the second main body part is equal to or more than 90° and equal to or less than 180°.

7. The magnetization device according to claim 2, wherein
the connection portion is expandable and contractible, and
a distance between the first end portion and the second end portion is adjusted by expanding and contracting the connection portion.

8. The magnetization device according to claim 3, wherein
the magnetization yoke includes:
a first main body part having the first end portion;
a second main body part having the second end portion; and
a connection portion that connects the first main body part and the second main body part opposite the first end portion and the second end portion, respectively, around which the magnetization coil is wound, and
an angle formed by the first main body part and the second main body part is equal to or more than 90° and equal to or less than 180°.

9. The magnetization device according to claim 3, wherein
the connection portion is expandable and contractible, and
a distance between the first end portion and the second end portion is adjusted by expanding and contracting the connection portion.

10. The magnetization device according to claim 4, wherein
the connection portion is expandable and contractible, and
a distance between the first end portion and the second end portion is adjusted by expanding and contracting.

11. The magnetization device according to claim 4, wherein the first main body part and the second main body part each include a narrower intermediate portion between the first end portion and second end portions and where the first and second main body part is connected to the connection part.

12. The magnetization device according to claim 11, wherein the narrower intermediate portions of the first main body part and the second main body part do not overlap along a direction in which the connection part extends.

13. The magnetization device according to claim 4, wherein the first main body part, the second main body part and the connection part are integral.

14. The magnetization device according to claim 2, wherein
the first accommodating part includes two sections, at least one of which is a movable section, and
the second accommodating part accommodating part includes two sections, at least one of which is a movable section.

15. The magnetization device according to claim 14, wherein
the movable section of the first accommodating part is rotatable, and
the movable section of the second accommodating part is rotatable.

16. The magnetization device according to claim 14, wherein
the movable section of the first accommodating part is translatable, and
the movable section of the second accommodating part is translatable.

17. The magnetization device according to claim 2, wherein the first accommodating part and the second accommodating part fully surround a periphery of the linear member.
